(19) United States
(12) Patent Application Publication
(10) Pub. No.: US 2024/0058503 A1
(43) Pub. Date: Feb. 22, 2024

(54) SPRAY-TYPE HYDROGEL

(71) Applicant: SEOUL NATIONAL UNIVERSITY R&DB FOUNDATION, Seoul (KR)

(72) Inventors: Nathaniel Suk-Yeon HWANG, Seoul (KR); Wonmoon SONG, Seoul (KR); Haein JEONG, Daejeon (KR); Seoyeon KIM, Seoul (KR)

(73) Assignee: SEOUL NATIONAL UNIVERSITY R&DB FOUNDATION, Seoul (KR)

(21) Appl. No.: 18/271,131

(22) PCT Filed: Jan. 10, 2022
(86) PCT No.: PCT/KR2022/000370

§ 371 Date: Jul. 6, 2023

(30) Foreign Application Priority Data
Jan. 8, 2021 (KR) 10-2021-0002724

(51) Int. Cl. A61L 26/00; C08L 5/08

(52) U.S. Cl. CPC A61L 26/008; A61L 26/0023; A61L 26/0052; A61L 26/0066; A61L 26/0076; C08L 5/08; C08L 2203/02

(57) ABSTRACT

According to one aspect of the present invention, there is provided a method for preparing a hydrogel, comprising: bringing into contact with each other (i) a positively charged composition comprising a positively charged polymer or a derivative thereof, and a first temperature-sensitive polymer; and (ii) a negatively charged composition comprising a negatively charged polymer and a second temperature-sensitive polymer.

### TECHNICAL FIELD

The present invention relates to a spray-type hydrogel.

### BACKGROUND ART

A hydrogel is a three-dimensional polymeric structure that can contain a large amount of water, and is characterized by physical properties similar to those of living tissue and excellent biocompatibility. In addition, the physical and chemical properties of hydrogels can be easily controlled by varying the type of constituent polymer, crosslinking agent, processing method, etc. Due to these excellent properties for use as biomaterials, various functional hydrogels (adhesives, hemostatic agents, drug carriers, cell therapy agents, artificial tissues, etc.) have been actively researched and developed.

The hydrogel is delivered to the target site by a method such as an injection-type method, a patch-type method, an implant-type method, or a spray-type method. The efficient delivery technology considering the application environment of the hydrogel is also important in addition to the functionality of the hydrogel itself. Despite the excellent functionality, there are cases in which problems occur during the application process of the hydrogel. For example, when implanted in the body, the hydrogel may not properly attach to the target site, and thus its original function may not be exhibited or may be lost, which may cause unexpected side effects.

Spray-type hydrogel delivery technology is a method of spraying a hydrogel through a spray device and applying it to a target area. Compared to delivery methods such as injection, patches, implantation, etc., it has the advantage that it can be quickly applied to a wider area. However, in order to successfully develop spray-type hydrogels, there is a limitation in that it is difficult to optimize the physical properties and conditions of materials. The materials used in the spray-type hydrogels should have reduced physical properties so as to separate into particles at the moment of spraying, while physical properties should be increased for stable attachment to the target site. Therefore, it is very important to select materials and optimize conditions that can satisfy the conflicting properties for delivery and attachment.

Due to the above technical difficulties, various commercially available spray-type hydrogels also have their own limitations. For example, AdSpray®, a spray-type anti-adhesion agent released in 2016 by Terumo Co., Ltd. in Japan, has limitations in that the powder should be made into a solution and applied to the body within an hour, gelation may fail due to mistakes in the manufacturing process, sprays that fail to undergo gelation are lost in the body and can cause inflammation, the use of the spray device is complicated and requires separate training, etc.

Therefore, there is a need for research and development of a spray-type hydrogel that can solve the above problems.

### DISCLOSURE

### Technical Problem

It is one object of the present invention to provide a hydrogel that is stable in a liquid phase and can be delivered to the body using various delivery systems such as a spray.

In addition, it is another object of the present invention to provide a spray-type hydrogel that exhibits an anti-inflammatory function and can be rapidly gelled in the body and applied topically to the application site.

### Technical Solution

According to one aspect of the present invention, there is provided a method for preparing a hydrogel, including: bringing into contact with each other (i) a positively charged composition including a positively charged polymer or a derivative thereof, and a first temperature-sensitive polymer; and (ii) a negatively charged composition including a negatively charged polymer and a second temperature-sensitive polymer.

Additionally, according to another aspect of the present invention, there is provided a method for treating a wound of a subject, including applying the prepared hydrogel to the wound.

Further, according to still another aspect of the present invention, there is provided a kit including: (i) a positively charged composition including a positively charged polymer or a derivative thereof, and a first temperature-sensitive polymer; and (ii) a negatively charged composition including a negatively charged polymer and a second temperature-sensitive polymer

Moreover, according to yet another aspect of the present invention, there is provided a use of the kit described above in the treatment of wounds of a subject or in the treatment of inflammation.

### Advantageous Effects

According to one embodiment of the present invention, there is provided a hydrogel that is stable in a liquid phase and can be delivered to the body using various delivery systems such as a spray, a powder, etc.

In addition, according to another embodiment of the present invention, there is provided a spray-type hydrogel that exhibits an anti-inflammatory function and can be rapidly gelled in the body and applied topically to the application site.

### BRIEF DESCRIPTION OF DRAWINGS

FIG. 1 is an overview of the hydrogel according to one embodiment of the present invention.

FIG. 2 is a chemical reaction scheme showing the synthesis of sulfated hyaluronic acid contained in the spray-type hydrogel according to one embodiment of the present invention and the regulation of the degree of substitution of sulfated group.

FIG. 3 is the result of evaluating the physical properties of the hydrogel according to one embodiment of the present invention.

FIG. 4 is the result of evaluating the physical properties of the hydrogel according to the solvent.

FIG. 5 is a graph of rheology data according to the concentration of chitosan.

FIG. 6 is a graph of rheology data according to pH.

FIG. 7 is an evaluation of physical properties of the hydrogel according to the concentration of Pluronic F127, a temperature-sensitive polymer.

FIG. 8 is the result of spraying to form hydrogel at 37° C.

FIG. 9 is the result of spraying to form hydrogel at 25° C.

### DETAILED DESCRIPTION OF PREFERRED EMBODIMENTS

The invention can make various modifications and take various forms, and thus specific embodiments are illustrated and described in detail below. It should be understood, however, that the invention is not intended to be limited to any particular disclosure form, but includes all modifications, equivalents, and alternatives falling within the spirit and scope of the invention.

In describing the drawings, similar reference numerals are used for similar elements. In the accompanying drawings, the dimensions of the structures are shown in an enlarged scale for clarity of the invention. Terms such as the first, the second, etc. may be used to describe various components for understanding, and the components should not be limited by the terms. The terms are used only for the purpose of distinguishing one component from another. For example, without departing from the scope of the present invention, a first component may be referred to as a second component, and similarly, the second component may also be referred to as the first component. Singular expressions include plural expressions unless the context clearly indicates otherwise.

In this application, it should be understood that terms such as “comprise/include” or “have” are intended to indicate that there is a feature, number, step, operation, component, part, or a combination thereof described in the specification, and they do not exclude in advance the possibility of the presence or addition of one or more other features or numbers, steps, operations, components, parts, or combinations thereof. Also, when a portion such as a layer, a film, an area, a plate, etc. is referred to as being “on” another portion, this includes not only the case where the portion is “directly on” the other portion but also the case where further another portion is interposed therebetween. Meanwhile, when a portion such as a layer, a film, an area, a plate, etc. is referred to as being “under” another portion, this includes not only the case where the portion is “directly under” the other portion but also the case where further another portion is interposed therebetween.

In the present specification, the terms “front side” and “back side” are used as relative concepts for easy understanding of the technical concept of the present invention. Therefore, the “front side” and “back side” do not refer to a specific direction, location, or component, but may be used interchangeably with each other. For example, the term “front side” may be interpreted as the term “back side”, and the term “back side” may be interpreted as the term “front side”. Thus, the term “front side” may be expressed as “first”, and the term “back side” may be expressed as “second”. Alternatively, the term “back side” may be expressed as “first”, and the term “front side” may be expressed as “second”. However, in a single embodiment, the terms “front side” and “back side” should not be used interchangeably.

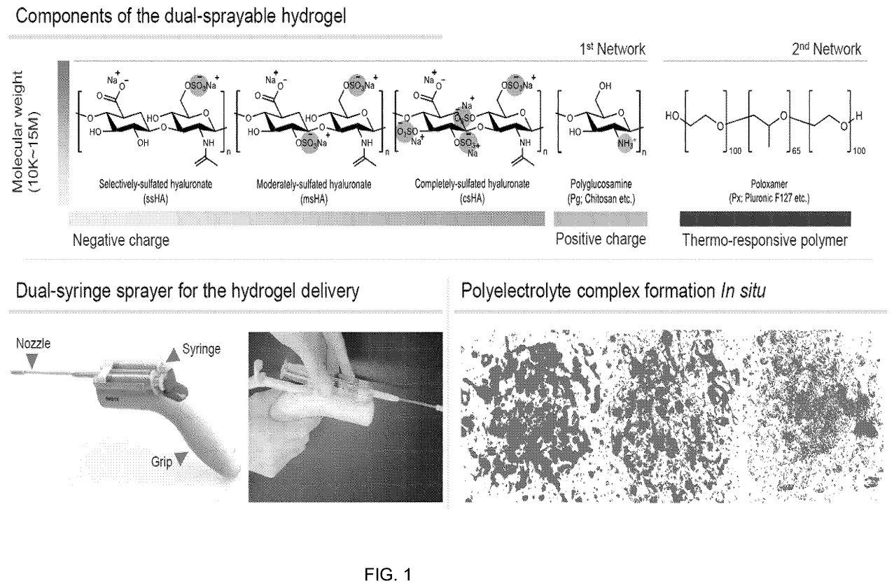

FIG. 1 is an overview of the hydrogel according to one embodiment of the present invention.

The hydrogel according to the present invention can be prepared by bringing into contact with each other (i) a positively charged composition including a positively charged polymer or a derivative thereof, and a first temperature-sensitive polymer; and (ii) a negatively charged composition including a negatively charged polymer and a second temperature-sensitive polymer.

The hydrogel according to the present invention consists of two parts, i.e., a positively charged composition including a positively charged polymer or a derivative thereof, and a first temperature-sensitive polymer; and (ii) a negatively charged composition including a negatively charged polymer and a second temperature-sensitive polymer. They can undergo gelation at body temperature.

The two solutions are injected through each syringe of a dual spray device, and as soon as the solutions come out of the syringe, they can be broken by an air pump connected to the device and sprayed in the form of a spray. As soon as the two sprayed solutions come into physical contact, they quickly form a bond and can settle on the target site. The poloxamer (e.g., Pluronic F127) in the settled solution mixture may be further gelled by body temperature to make the hydrogel more stable.

One of the limitations in the use of temperature-sensitive polymers is that gelation time by body temperature may be prolonged. For example, in the case of conventional anti-adhesion agents, even when gelation of poloxamer is used, it usually takes about 30 seconds for gelation by body temperature. Accordingly, there have been cases where side effects have occurred due to such a slow gelation rate. Specifically, non-gelled poloxamer flows down and fails to sufficiently coat the surgical site, or pools in other non-target sites to cause inflammation.

However, since the hydrogel according to the present invention is based on the formation of a complex by electrostatic attraction, gelation is much faster than conventional products. In addition, since the gel network is not covalently bonded, components are gradually released over time after application to the surgical site. When macrophages absorb sulfated hyaluronate, NF-κB signaling is suppressed, causing M2 polarization. In other words, it can increase the therapeutic effect caused by anti-inflammatory effect in the surgical site.

The negatively charged polymer may be, for example, negatively charged hyaluronic acid and its derivatives. The negatively charged polymer may be, for example, at least one or more selected from the group consisting of polyacrylate, polystyrene sulfonate, alginate, chondroitin sulfate, heparin, and heparan sulfate.

The negatively charged hyaluronic acid may include sulfated hyaluronate. The hyaluronic acid may be obtained from a microorganism of the genus Streptococcus. Next, sulfated hyaluronic acid may be synthesized by substituting at least some of the hydroxyl groups in the molecule of the obtained hyaluronic acid with sulfated groups.

The negatively charged hyaluronic acid may include at least one sulfated hyaluronic acid selected from the following groups:

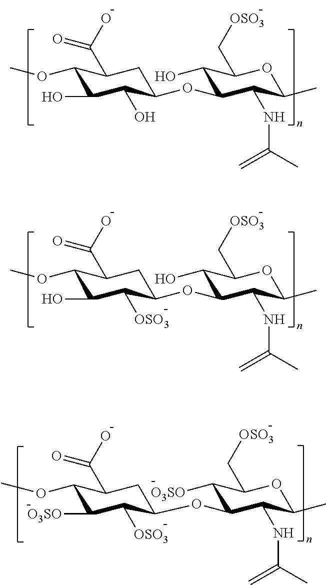

wherein n is a natural number from 20 to 5000. In some cases, n may be a natural number of 50, 100, 200, 300, 400, 500, 600, 700, 800, 1000, 2000, or 3000 or more, or 4000, 3000, 2000, 1000, 900, 800, 700, 600, or 500 or less. The hyaluronic acid may have a molecular weight of 20 kDa to less than 100 kDa, or in some cases, 100 kDa to 200 kDa, or 100 kDa to less than 1000 kDa, or greater than 1000 kDa.

The first and second temperature-sensitive polymers included in the hydrogel may include a poloxamer, and the poloxamer may be, for example, Pluronic F127, etc. The first temperature-sensitive polymer and/or the second temperature-sensitive polymer may include a compound having the following structure:

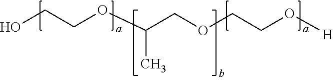

wherein a is a natural number from 2 to 130, and b is a natural number from 15 to 67. In some cases, a is a natural number from 90 to 110. In some cases, b is a natural number from 55 to 67.

In some cases, the first temperature-sensitive polymer and/or the second temperature-sensitive polymer may include a compound having the following structure:

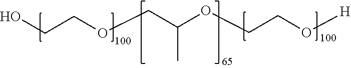

The first temperature-sensitive polymer and/or the second temperature-sensitive polymer may have a molecular weight of 10 kDa to 15 kDa.

The positively charged polymer may be polyglucosamine, polyethyleneimine, or a positively charged biopolymer substituted with n-substituted amine. For example, the positively charged polymer may be at least one selected from the group consisting of polyglucosamine, poly(L-lysine), polyethyleneimine, poly(lactic-co-glycolic acid), poly(ester amine)s, poly(2-(dimethylamino)ethyl methacrylate), and poly(amidoamine) dendrimers. In some cases, the positively charged polymer may include polyglucosamine, and the polyglucosamine may be chitosan, etc. The chitosan may have a molecular weight of 50 kDa to 335 kDa or, in some cases, a molecular weight of 120 kDa to 250 kDa. In some cases, it may have a molecular weight of 50 kDa to less than 190 kDa (LMW), 190 kDa to less than 310 kDa (MMW), or 310 kDa to 375 kDa (HMW). In addition, the positively charged composition may include 1.2 wt. % to 2.0 wt. % chitosan, or in some cases 1.6 wt. % to 2.0 wt. % chitosan.

The positively charged composition may further include hydrochloric acid (HCl), or the solvent provided to the positively charged composition may only be hydrochloric acid (HCl). The positively charged composition may have an acidity of pH 4.7 to pH 6.0, or in some cases pH 5.2 to pH 6.0. The positively charged composition may not include acetic acid.

The positively charged composition and the negatively charged composition may be mixed after being released from the spray-type delivery device. At this time, the negatively charged hyaluronic acid included in the mixture forms a primary electrostatic network with the mixed polyglucosamine. Since the primary electrostatic network is formed by the electrostatic attraction between the negatively charged hyaluronic acid and the positively charged polyglucosamine, it can be formed immediately after mixing the positively charged composition and the negatively charged composition.

When examining the primary electrostatic network formation more closely, the negatively charged hyaluronic acid (e.g., sulfated hyaluronate) has a strong negative charge due to its carboxy group and multiple sulfated groups, whereas polyglucosamine has a positive charge due to its amine group. Therefore, when two polymers are mixed, a bond between the two polymers is instantaneously formed by electrostatic interaction. Since this is a reversible bond, destruction and regeneration of intermolecular bonds occur when a physical shock is applied from the outside, and based on such characteristics, it may exhibit unique rheological behaviors such as self-renewal ability, shock absorption ability, gel-sol state change, etc.

Therefore, according to one embodiment of the present invention, since the cross-linking system by electrostatic interaction between negatively charged hyaluronic acid and polyglucosamine is utilized, a method of delivering the gel into the body in the form of an injection by filling the mixture into a syringe; a method of delivering the gel into the body in the form of a spray by simultaneously spraying the two solutions separately and mixing them in an aerosol state in the air; and a method of gelation by delivering in the form of a powder as it absorbs the body fluid may all be used. The versatility of the present invention can provide excellent convenience for the delivery of a hydrogel to a living body.

Next, the formed primary electrostatic network forms a secondary gelation network with a temperature-sensitive polymer (e.g., poloxamer). The secondary gelation network may be formed in a temperature-sensitive manner. For example, after the primary electrostatic network and the temperature-sensitive polymer are attached to the application site in the body, a cross-linking reaction for forming a secondary gelation network may proceed due to body temperature. Since the secondary gelation network is not formed by electrostatic attraction but by chemical reaction, unlike the primary electrostatic network, it may not be formed immediately, and activation energy may be required to initiate the reaction. Activation energy for reaction initiation can be supplied from body temperature.

Poloxamers such as Pluronic F127 maintain a liquid state at low temperatures, but have the property that they undergo a reversible gelation at temperatures in the range of body temperature. Such a temperature-sensitive polymer has a disadvantage in that gelation takes about 30 seconds. If the poloxamer does not stay on the application site before gelation occurs and flows down, the original intended function may not be achieved, and it may flow into other organs and cause unwanted reactions such as inflammatory reactions. For example, Mediclore® from CG BIO according to the prior art is an injectable anti-adhesion agent based on gelatin, chitosan, and temperature-sensitive poloxamer. When a liquid product is administered into the body, it undergoes gelation by body temperature. As discussed above, this product has the disadvantage in that the gelation of poloxamer is slow (within 30 seconds), and the liquid product flows into the organs before gelation occurs.

Since the hydrogel system undergoes primary gelation within a few seconds immediately after spraying, Pluronic F127 does not flow down at the application site and can be fixed and induce secondary gelation. In the case of the present invention, as the spray-type hydrogel forms a secondary gelation network after the rapid formation of a primary electrostatic network, it can be quickly applied topically without flowing down from the application site.

The spray-type hydrogel may exhibit an anti-inflammatory function. In the case of hydrogels to be inserted into the body, there was a problem in that side effects caused by inflammation frequently occurred. However, since the spray-type hydrogel according to one embodiment of the present invention exhibits an anti-inflammatory function, there is no risk of causing inflammation.

The contact may be performed at room temperature and may be performed in a form in which the hydrogel is sprayed onto the wound. At this time, the wound may be a surgical site, and after spraying the hydrogel onto the wound, inflammation of the wound may be treated. The target of wound treatment may be a human in some cases.

Meanwhile, in regard to the treatment using a spray, it is possible to expand the range of use by supporting a physiologically active substance in a hydrogel. For example, the range of use of the spray-type hydrogel can be expanded by supporting/delivering various flavonoid-based substances with low solubility in water, such as amentoflavone, quercetin, phloretin, etc., or drugs with functions such as anti-inflammation and tissue regeneration, etc. In particular, the hydrogel according to the present invention can be used by supporting the above-mentioned various physiologically active substances because the temperature-sensitive polymer (e.g., poloxamer) has amphiphilic properties and is suitable for supporting hydrophobic substances.

The technical principle of the spray-type hydrogel according to the present invention is as follows.

### 1. Synthesis of Sulfated Hyaluronic Acid and Regulation of Degree of Substitution of Sulfated Group

The solubility for the organic solvent is increased by substituting the sodium of the sodium salt of hyaluronic acid with tetrabutylammonium, and then the resultant is dissolved in dimethylformamide and treated with a sulfated pyridine salt at 40° C. to obtain sulfated hyaluronate. In particular, the degree of substitution of the sulfated group can be regulated by changing the amount of the pyridine sulfate salt and the treatment time. Among the hyaluronic acid units, the hydroxyl group bonded to carbon 6 of N-acetylglucosamine can easily rotate sterically and thus can react the fastest, and the hyaluronic acid whose reaction is terminated at this stage is called sulfated hyaluronic acid (degree of substitution=1). Among the hyaluronic acid units, the hydroxyl group bound to carbon 2 of glucuronic acid reacts next, and the hydroxy group bound to carbon 3 of glucuronic acid and carbon 4 of N-acetylglucosamine react simultaneously under subsequent conditions.

Among the hyaluronic acid units, the hydroxyl group bound to carbon 2 of glucuronic acid reacts next, and under the following conditions, the hydroxy group bound to carbon 3 of glucuronic acid and carbon 4 of N-acetylglucosamine reacts simultaneously.

Depending on the degree of substitution of the sulfated group, the physicochemical properties (solubility, negative charge) of sulfated hyaluronic acid vary, and accordingly, biocompatibility varies. As the substitution rate of the sulfated group increases, the negative charge of hyaluronic acid gets stronger, and thus the cohesive force for positively charged polymers such as polyglucosamine increases. Further, in addition to the existing CD44-mediated signaling ability of hyaluronic acid, when delivered in vivo, it binds to cytokines and growth factors, increasing the ability to suppress inflammation or angiogenesis, and the interaction with hyaluronidase decreases, thus increasing stability in the body. The existing hyaluronic acid is rapidly decomposed within a few hours or days when introduced into the body, and thus, even if it is attached to the body, it loses its function before the wound is regenerated.

### 2. Cross-Linking Between Sulfated Hyaluronic Acid and Polyglucosamine by Electrostatic Interaction

Sulfated hyaluronic acid has a strong negative charge due to its carboxyl group and multiple sulfated groups, whereas polyglucosamine has a positive charge due to its amine group. Therefore, when two polymers are mixed, a bond between the two polymers is instantaneously formed by electrostatic interaction. Since this is a reversible bond, destruction and regeneration of intermolecular bonds occur when a physical shock is applied from the outside, and based on such characteristics, it may exhibit unique rheological behaviors such as self-renewal ability, shock absorption ability, gel-sol state change, etc.

Therefore, if the crosslinking system by electrostatic interaction between the sulfated hyaluronic acid of the present invention and polyglucosamine is utilized, the two solutions are individually sprayed at the same time and mixed in an aerosol state in the air, thereby delivering the gel to the body in the form of a spray. The spray-type hydrogel can be quickly applied to a wide area and thus is expected to be efficiently introduced to various diseases with a wide range of symptoms such as adhesions after surgery, burns, etc.

### 3. Gelation of Temperature-Sensitive Poloxamer by Body Temperature

Poloxamers such as Pluronic F127 maintain a liquid state at low temperatures, but have the property of undergoing a reversible gelation at temperatures in the range of body temperature. Such a temperature-sensitive polymer has a disadvantage in that gelation takes about 30 seconds. When applied to the body, if the poloxamer does not stay on the application site before gelation occurs and flows down, the original intended function may not be achieved, and it may flow into other organs and cause unwanted reactions such as inflammatory reactions. Since the hydrogel system undergoes primary gelation within a few seconds immediately after spraying, Pluronic F127 does not flow down at the application site and can be fixed and induce secondary gelation.

Next, when examining the spray-type hydrogel delivery device more closely, the delivery device may be provided in the form of a dual syringe spray, a miniaturized spray, an injectable syringe, a spray-type powder, etc.

In the case of a dual syringe spray, when the syringe is pressed, the primary gelling mixture and polyglucosamine are simultaneously released, and the ejected liquids are bonded to each other because the distance between the nozzles is very close. In this process, the compressed air in the middle mixing section may spray the solution mixing section in the form of droplets. Therefore, the primary gelling mixture and polyglucosamine may be provided in the form of droplets while being mixed.

In the case of a miniaturized spray, the problem associated with the dual syringe spray is improved. Due to the close distance between the nozzles of the dual syringe spray, if the concentration of the mixed solution increases, strong gelation occurs before spraying, and it may flow without being sprayed. In addition, it is inconvenient that an air compressor is required. The miniaturization sprays have ameliorated such problems, and they were designed so that the distance between the nozzles could be spaced apart and the mixture could be sprayed with only hand pressure.

In the case of an injectable syringe, since sulfated hyaluronic acid and polyglucosamine are bonded through an electrostatic interaction, the reversible destruction and regeneration of the network is considered. The injectable syringe may be provided in the form of a single syringe as well as a dual syringe injectable system, and it is possible to deliver gel in an injectable form.

The spray-type powder provides sulfated hyaluronate/polyglucosamine in powder form. Sulfated hyaluronic acid/polyglucosamine mixture in powder form is activated by body fluids when applied to the target area, and thus can exhibit anti-adhesion and hemostatic effects.

The spray-type hydrogel delivery device according to one embodiment of the present invention may be provided in the form of a finished product in which the primary gelling mixture and polyglucosamine are each buffered in the device. Therefore, it is possible to increase the surgical efficiency without the consumption of manpower coming from having someone in charge of preparing the solution during surgery.

The spray-type hydrogel has the advantage that it can be delivered to the body by way of various delivery methods. In addition, the anti-inflammatory and anti-angiogenic function of the components can be used for inflammatory diseases including anti-adhesion agents and in cosmetics. By delivering the gel to the joint through the hydrogel injection system, it can be applied to the treatment of joint diseases such as arthritis. In the case of a spray-type system, it can be applied to the field of cosmetics by utilizing the moisturizing and anti-inflammatory effects of the sulfated hyaluronic acid and the increase in gel retention due to instant gelation, etc.

Next, the method of preparing sulfated hyaluronic acid included in the spray-type hydrogel will be examined more closely.

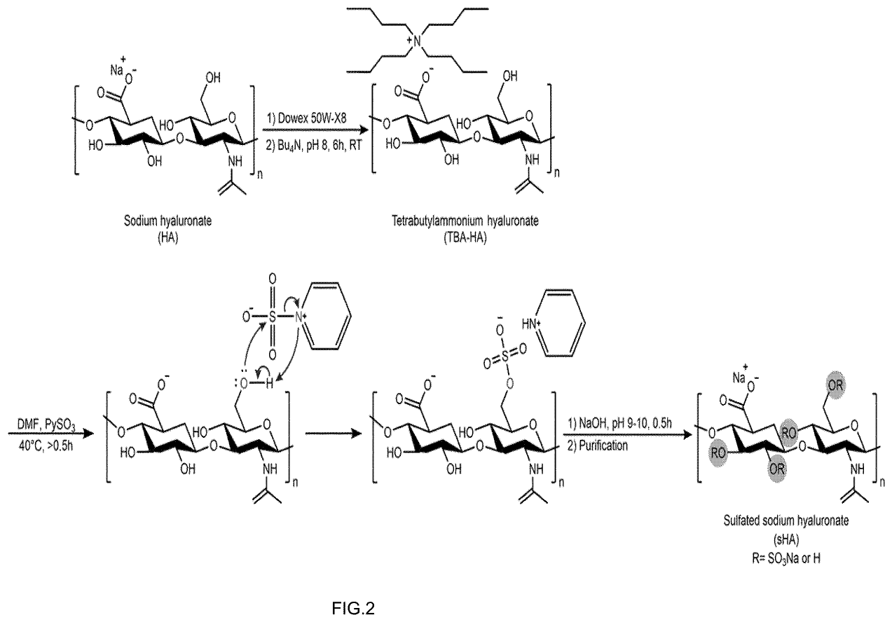

FIG. 2 is a chemical reaction scheme showing the synthesis of sulfated hyaluronic acid included in the spray-type hydrogel according to one embodiment of the present invention and the regulation of the degree of substitution of sulfated group.

Specifically, the sulfated hyaluronic acid can be obtained by substituting the sodium salt of the sodium salt of hyaluronic acid with tetrabutylammonium to increase solubility for organic solvents, and then dissolving in dimethylformamide and treating with sulfate pyridine salt at 40° C. In particular, the degree of substitution of the sulfated group can be regulated by changing the amount of the pyridine sulfate salt and the treatment time. Among the hyaluronic acid units, the hydroxyl group bonded to carbon 6 of N-acetylglucosamine can easily rotate sterically and thus can react the fastest, and the hyaluronic acid whose reaction is terminated at this stage is referred to as sulfated hyaluronic acid (degree of substitution=1). Among the hyaluronic acid units, the hydroxyl group bound to carbon 2 of glucuronic acid reacts next, and the hydroxy group bound to carbon 3 of glucuronic acid and carbon 4 of N-acetylglucosamine react simultaneously under subsequent conditions.

Each of the above products is referred to as sulfated hyaluronic acid (degree of substitution=2) and sulfated hyaluronic acid (degree of substitution=3).

Referring to Table 1 below, it can be confirmed that the degree of substitution of the sulfated group changes by regulating the sulfur trioxide pyridine complex and reaction time.

TABLE 1

| | | | Sulfur | | |
|---|---|---|---|---|---|
| | | | trioxide | | |
| | | | pyridine | | |
| Degree of sulfation | HA-TBA | DMF | complex | Reaction | Temperature |
| per repeating unit (%) | (mg) | (mL) | (mg) | time (h) | (° C.) |
| 25 (Degree of | 100 | 10 | 126 | 0.5 | 40 |
| substitution = 1) | | | | | |
| 50 (Degree of | | | 336 | 1 | |
| substitution = 2) | | | | | |
| 100 (Degree of | | | 504 | 3 | |
| substitution = 3) | | | | | |

Depending on the degree of substitution of the sulfated group, the physicochemical properties (solubility, negative charge) of sulfated hyaluronic acid vary, and accordingly, biocompatibility varies. As the substitution rate of the sulfated group increases, the negative charge of hyaluronic acid gets stronger, and thus the cohesive force for positively charged polymers such as polyglucosamine increases. Further, in addition to the existing CD44-mediated signaling ability of hyaluronic acid, when delivered in vivo, it binds to cytokines and growth factors, increasing the ability to suppress inflammation or angiogenesis, and the interaction with hyaluronidase decreases, thus increasing stability in the body. The existing hyaluronic acid is rapidly decomposed within a few hours or days when introduced into the body, and thus, even if it is attached to the body, it loses its function before the wound is regenerated. That is, although it is difficult to apply hyaluronic acid itself to a spray-type hydrogel, properties suitable for the spray-type hydrogel can be imparted by introducing a sulfated group.

Next, the advantageous effects of the spray-type hydrogel according to one embodiment of the present invention will be confirmed through the Examples below.

The experimental method performed to prove the advantageous effects in the present invention is as follows.

### 1. Summary of Terms

In the present invention, when the molecular weight of sulfated hyaluronic acid was less than 20 kDa, it was named “Oligo”, when it was 20 kDa to less than 100 kDa, it was named “LMW”, when it was 100 kDa to less than 1000 kDa, it was named “MMW”, and when it exceeded 1000 kDa, it was named “HMW”.

In addition, with respect to the degree of sulfation, when the substitution rate of —OH to —SO³⁻ was 12.5% to 37.5%, it was defined as the degree of sulfation 1, when the substitution rate was 37.5% to 62.5%, it was defined as the degree of sulfation 2, and when the substitution rate was 62.5% to 87.5%, it was defined as the degree of sulfation 3.

In addition, when the molecular weight was less than 50 kDa to 190 kDa, it was named “LMW”, when the molecular weight was 190 kDa to less than 310 kDa, it was named “MMW”, and when the molecular weight was 310 kDa to 375 kDa, it was named “HMW”.

### 2. Spray Test: Chitosan/F127 Solution

In the chitosan/F127 (solvent: 0.1 M HCl) mixed solution, the molecular weight of chitosan (LMW, MMW, HMW), the concentration of chitosan (1.2-2.0%), the concentration of F127 (14-19%), and the pH (5.2-6.0) were adjusted to prepare samples. The solution was loaded in an amount of 0.8 mL into an air pump-based spray and actuated by pressing the syringe at a rate of 9 mL/min. The sprayed solution was dispersed in a spray form by an air pressure of about 150 kPa. The dispersed gel was attached on a plate heated to 37° C. The spraying degree was quantitatively evaluated on the basis of 0-3 points, and the gelation degree was evaluated on the basis of 0-4 points. The criteria are as follows.
1) Spray degree (0 points: not sprayed/1 point: sprayed unevenly due to internal pressure/2 points: sprayed evenly even though pressure was applied/3 points: sprayed evenly without pressure).
2) Degree of gelation (0 points: no viscosity, no gelation, solution flows down/1 point: weak viscosity, solution flows down/2 points: has viscosity, but it takes a long time to gel (more than 30 seconds)/3 points: strong viscosity and gels immediately after spraying/4 points: strong viscosity but gels at room temperature).

### 3. Spray Test: Chitosan/F127 Solution and Sulfated Hyaluronic Acid/F127 Solution

The condition of the chitosan/F127 solution was fixed to chitosan (MMW; 2%)/F127 (16%)/pH 5.8/solvent: 0.1 M HCl. The condition of the sulfated hyaluronic acid/F127 solution was fixed to F127 concentration of 16%, solvent PBS (pH 7.4), and samples were prepared by adjusting the molecular weight (Oligo, LMW, MMW, HMW), degree of sulfation (1-3), and concentration (1.2-4.0%). Each solution was loaded in an amount of 0.8 mL into an air pump-based spray and actuated by pressing the syringe at a rate of 9 mL/min. The sprayed solution was dispersed in a spray form by an air pressure of about 150 kPa. The dispersed gel was attached on a plate heated to 37° C. The spraying degree was quantitatively evaluated on the basis of 0-3 points, and the gelation degree was evaluated on the basis of 0-4 points. The criteria are as follows.
1) Spray degree (0 points: not sprayed/1 point: sprayed unevenly due to internal pressure/2 points: sprayed evenly even though pressure was applied/3 points: sprayed evenly without pressure).
2) Degree of gelation (0 points: no viscosity, no gelation, solution flows down/1 point: weak viscosity, solution flows down/2 points: has viscosity, but it takes a long time to gel (more than 30 seconds)/3 points: strong viscosity and gels immediately after spraying/4 points: strong viscosity but gels at room temperature).

### 4. Measurement of Viscosity of Chitosan/F127 Solution

In the chitosan/F127 (solvent: 0.1 M HCl) mixed solution, the molecular weight of chitosan (LMW, MMW, HMW), the concentration of chitosan (1.2-2.0%), the concentration of F127 (14-19%), and the pH (5.2-6.0) were adjusted to prepare samples. The viscosity of the prepared samples was measured using the Advanced Rheometric Expansion System (ARES) manufactured by TA Instruments. 300 μL of the chitosan/F127 mixed solution was loaded onto an 80 mm diameter flat plate of ARES with a thickness of 0.6 mm, and then the viscosity (Pa·s) was recorded in the range of shear rate of 10 s⁻¹ to 1000 s⁻¹. The corresponding data was visualized in two types of graphs (viscosity value of the sample for the total shear rate and viscosity value of the sample at the shear rate of 1000 s⁻¹) through GraphPad Prism 8.0.1.

### 5. Measurement Rheology of Physical Properties of Chitosan/F127+Sulfated Hyaluronic Acid/F127 Mixed Solution (Frequency Sweep)

The condition of the chitosan/F127 solution was fixed to chitosan (MMW; 2%)/F127 (16%)/pH 5.8/solvent: 0.1 M HCl. The condition of the sulfated hyaluronic acid/F127 solution was fixed to F127 concentration of 16%, solvent PBS (pH 7.4), and samples were prepared by adjusting the molecular weight (Oligo, LMW, MMW, HMW), degree of sulfation (1-3), and concentration (1.2-4.0%). The chitosan/F127 solution and the sulfated hyaluronic acid/F127 were mixed in a ratio of 1:1, and 300 μL of gel was loaded onto an 80 mm diameter flat plate of ARES with a thickness of 0.6 mm. Frequency sweep was performed on the loaded samples. G′ and G″ were measured under the following conditions: 5% strain rate, angular frequency 0.1-100 ω at 25° C. Data were visualized in two types of graphs (Frequency sweep, Tangent) through GraphPad Prism 8.0.1. Upon mixing the two solutions, the final composition of the gel became chitosan (MMW; 1%)/F127 (16%)/pH 5.8/sulfated hyaluronic acid (Oligo-HMW, degree of sulfation 1-3, concentration 0.6-2.0%).

In order to solve the problem of gelation delay, which is seen in conventional temperature-sensitive polymers, optimization of chitosan viscosity and poloxamer concentration was performed.

Chitosan is an element that is difficult to be optimized in the hydrogel kit according to the present invention. In order for the spray to be sprayed, the viscosity must be low, but the general chitosan solution has a high viscosity, making it unsuitable for use as a spray. Accordingly, the optimum viscosity was obtained by changing various conditions of the chitosan solution. The factors considered to optimize the viscosity of chitosan are as follows: 1) type of solvent—viscosity is decreased when dissolved in hydrochloric acid, 2) molecular weight—chitosan with a molecular weight of 340 kDa is too viscous to be used as a spray, 3) viscosity is decreased when pH is lower than 6, and 4) concentration—chitosan with a concentration of 2.0% or more has too high a viscosity to be used as a spray.

In the present invention, it was designed such that 1.2 wt. % to 2.0 wt. % chitosan is included in the positively charged composition, or in some cases, 1.6 wt. % to 2.0 wt. % chitosan is included in the positively charged composition.

Additionally, it was designed such that the solvent included in the positively charged composition includes hydrochloric acid (HCl) or consists only of hydrochloric acid (HCl).

Further, it was designed such that the positively charged composition has an acidity of pH 4.7 to pH 6.0, or in some cases, an acidity of pH 5.2 to pH 6.0.

In addition, concentration optimization of Pluronic F127, a type of temperature-sensitive polymer, was performed. The concentration conditions are important because poloxamers cause physical crosslinking by temperature. If the concentration is too low, poloxamers cannot sufficiently come into contact with each other, and if the concentration is too high, they may form a gel at room temperature.

In the present invention, the positively charged composition was designed to include the first temperature-sensitive polymer in an amount of 16% to 17%, and/or the negatively charged composition was designed to include the second temperature-sensitive polymer in an amount of 16% to 17%. In addition, the hydrogel was designed such that the total amount of the first temperature-sensitive polymer and the second temperature-sensitive polymer was less than 18%.

TABLE 2

| | | Present |
|---|---|---|
| | Prior Art | Invention |
| Solvent | Acetic Acid | HCl |
| Molecular Weight (kDa) | High | 120, 250, 340 |
| pH | 4.65 | 5.2-6.0 |
| Chitosan Concentration (%) | 2.0 | 1.2-2.0 |
| Concentration of Temperature- | 18 | 12-17 |
| Sensitive Polymer (%) | | |

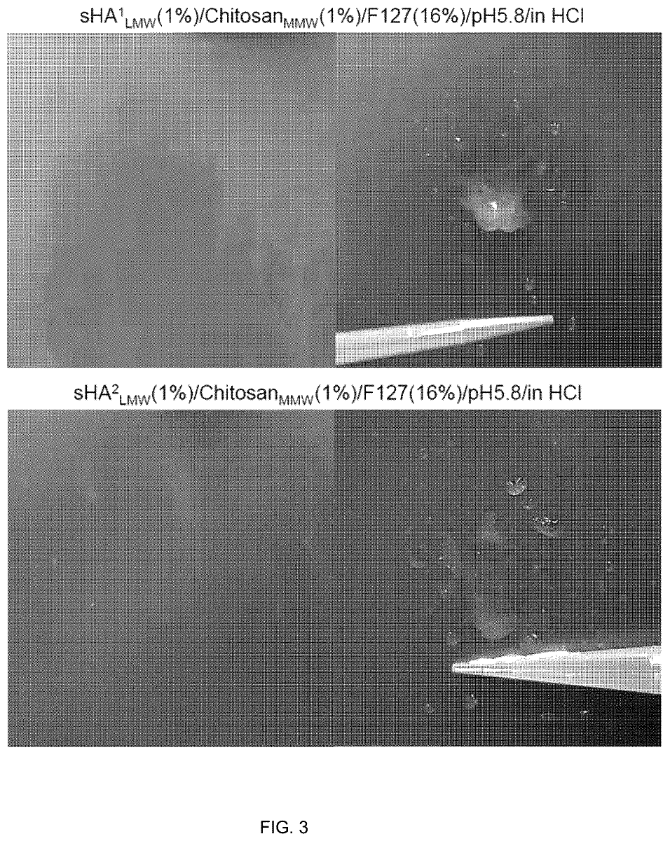

FIG. 3 is the result of evaluating the physical properties of the hydrogel according to one embodiment of the present invention.

FIG. 3 is the result of spraying each mixture of sHA¹_{LMW} (1%)/Chitosan_{MMW} (1%)/F127 (16%)/pH 5.8/HCl solvent (FIG. 2 left) and sHA²_{LMW} (1%)/Chitosan_{MMW} (1%)/F127 (16%)/pH 5.8/HCl solvent. The two mixtures disclosed in FIG. 2 differ only in the degree of sulfation of hyaluronic acid.

The basic composition of the spray-type hydrogel can be determined according to 1) the degree of sulfation of sulfated hyaluronic acid (1 to 3), 2) the molecular weight of sulfated hyaluronic acid (LMW, MMW, HMW), and 3) the molecular weight of chitosan (LMW, MMW, HMW).

Referring to the drawing, it can be confirmed that gelation proceeded immediately after spraying when the degree of sulfation of the sulfated hyaluronic acid was 1 and 2.

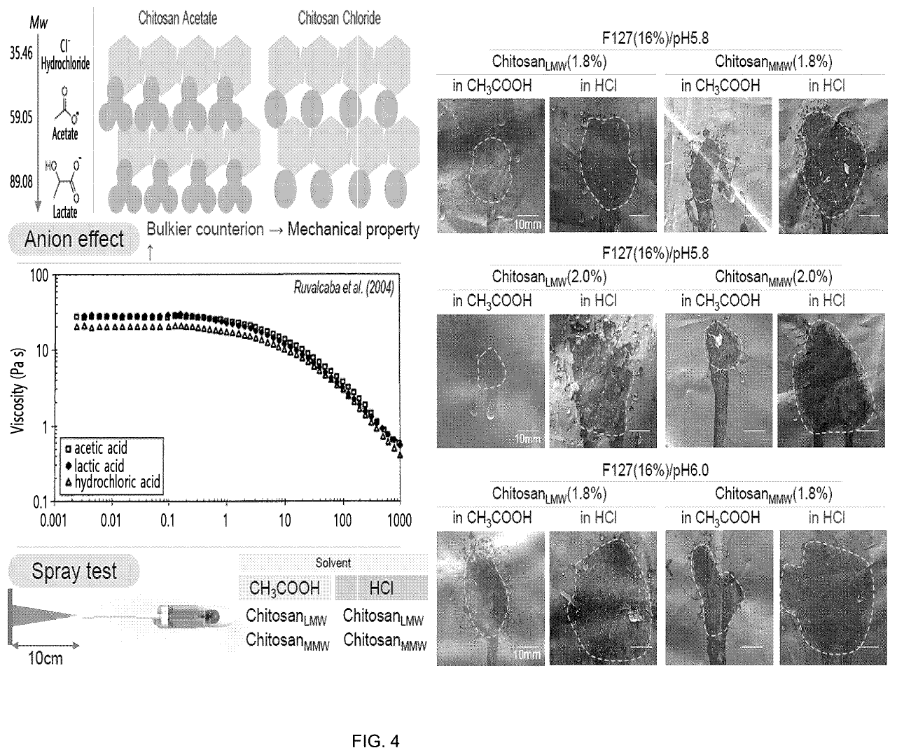

FIG. 4 is the result of evaluating the physical properties of the hydrogel according to the solvent.

Referring to FIG. 4, the gelation and spraying patterns of the hydrogel were confirmed when an acetic acid solvent was used and when HCl was used.

If the viscosity of the solution is too high, spraying is impossible, and the solution is not spread evenly, but is instead ejected as if spitting out chunks. When chitosan is dissolved in acetic acid (1% in D.W.), the viscosity becomes too high to be suitable as a spray. When chitosan is dissolved in HCl (0.1 M), the viscosity is significantly lowered. This is known as a difference in physical properties depending on the size of acetate and chloride ion. As a result of a spray test by slowly mixing NaOH with the chitosan/F127 (15%) solution while adjusting the pH to 5.6 to 6.0, the chitosan/F127 (15%) solution dissolved in HCl had an appropriately low viscosity, and there was no particular problem with spraying. However, the chitosan/F127 (15%) solution dissolved in acetic acid had a high viscosity and was ejected in chunks from the spray. That is, it was not spread widely (i.e., very narrow spray angle) with the spray. Therefore, it was confirmed that it is appropriate to use HCl as the chitosan solvent.

Next, the physical properties of the hydrogel were evaluated according to the concentration of chitosan, a type of polyglucosamine.

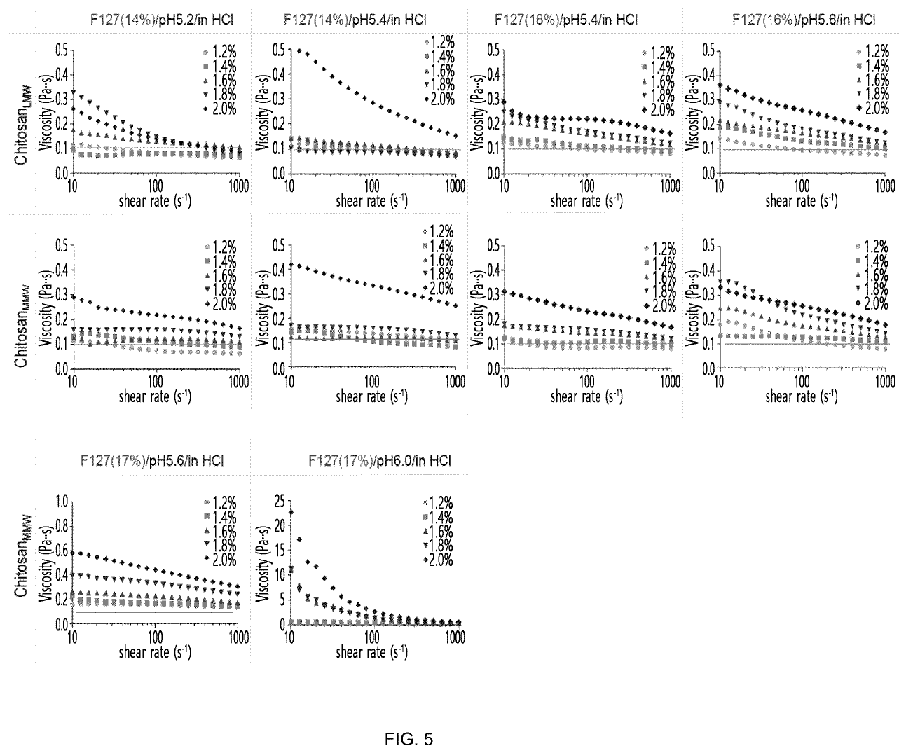

FIG. 5 is a graph of rheology data according to the concentration of chitosan.

Regarding the concentration of chitosan, as the concentration of the solution increases, the viscosity increases because entanglement of the chitosan polymer occurs. The viscosity of the solution required for spraying is known to be approximately 0.1 Pa·s, and if it is lower than that, the viscosity becomes too low, causing damage to the device by generating turbulence in the spray device, or it may be sprayed onto a wider area than the target, making it messy, and may flow down, etc. In the case of using a solution having a viscosity higher than 0.1 Pa·s, it is difficult to spray the solution as the viscosity reaches 0.5 Pa·s. In other words, 0.1 Pa·s is said to be the optimum viscosity for spraying. Since the shear rate applied to the solution when the solution is sprayed is from 1000 s⁻¹ to 40000 s⁻¹, in the present invention, while measuring the viscosity with a rheometer, it was confirmed whether the viscosity of the solution reached 0.1 Pa·s when the shear rate was 1000 s⁻¹. In addition, the shear thinning phenomenon, in which the viscosity decreases as the stress applied to the solution increases, that is, as the shear rate increases, was also set as an optimal condition. A solution showing a shear thinning pattern recovers its viscosity after spraying when the stress applied to the solution is removed. A low-viscosity solution is preferred when used as a spray, but the gel can be stably maintained when the viscosity is high after being attached to the surgical site, and thus, sufficient shear thinning was considered the optimal condition.

Based on the rheology data, the optimal concentration condition for chitosan was narrowed down to 1.6% or more. The upper part of the drawing (Chitosan_{LMW}) is the viscosity of low-molecular-weight chitosan/F127 (16%) measured by rheology, and as the shear rate from 10 s⁻¹ to 1000 s⁻¹ is applied to the solution, it can be seen that the viscosity gradually decreases. The group with the lowest concentration of chitosan (1.2-1.4%) showed a viscosity of 0.1 Pa·s with almost no shear thinning pattern, but rather flat, and the viscosity decreased beyond 0.1 Pa·s at 1000 s⁻¹. In this case, the viscosity was too low to maintain the shape after reaching the target point and flowed down, and accordingly, it did not meet the spray conditions and was excluded from the concentration range. When the concentration of chitosan exceeded 2.0%, the viscosity increased, and above all, whenever NaOH was added in the pH control step, strong precipitation of chitosan is occurred, so that chitosan did not dissolve no matter how much it was dissolved. That is, it was concluded that the optimum concentration of chitosan should not be higher than 2.0% for handling reasons. As a result, the optimum concentration of chitosan required for spraying is 1.6-2.0%.

Next, the physical properties of the hydrogel according to pH were evaluated.

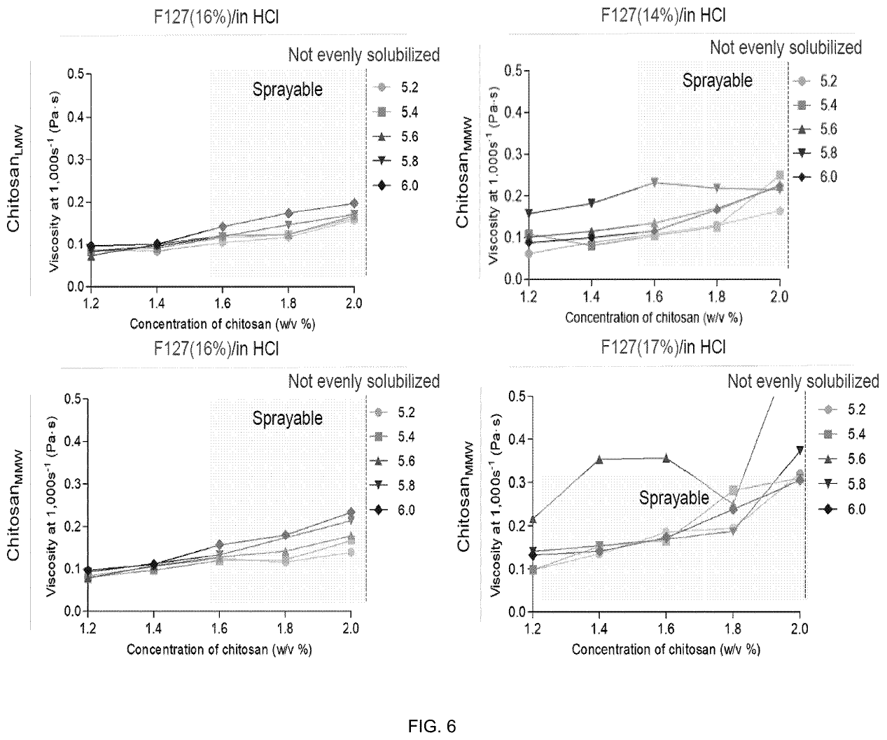

FIG. 6 is a graph of rheology data according to pH.

The pH of the chitosan had virtually no significant effect on the spray. As the pH increased, the viscosity increased, but the pressure applied by the spray device was strong enough to instantly lower the viscosity of the chitosan solution. However, considering that the pH of the commonly used peritoneal dialysis fluid is 5.2 and that cytotoxicity may occur due to acidic pH, it was determined that 5.2 is appropriate for the lower limit of pH. Under the condition of pH 6.0 or higher, since the chitosan solution was strongly precipitated and time was required to prepare the solution, the appropriate pH condition was set to 5.2-6.0.

Next, the physical properties of the hydrogel were evaluated according to the concentration of the temperature-sensitive polymers.

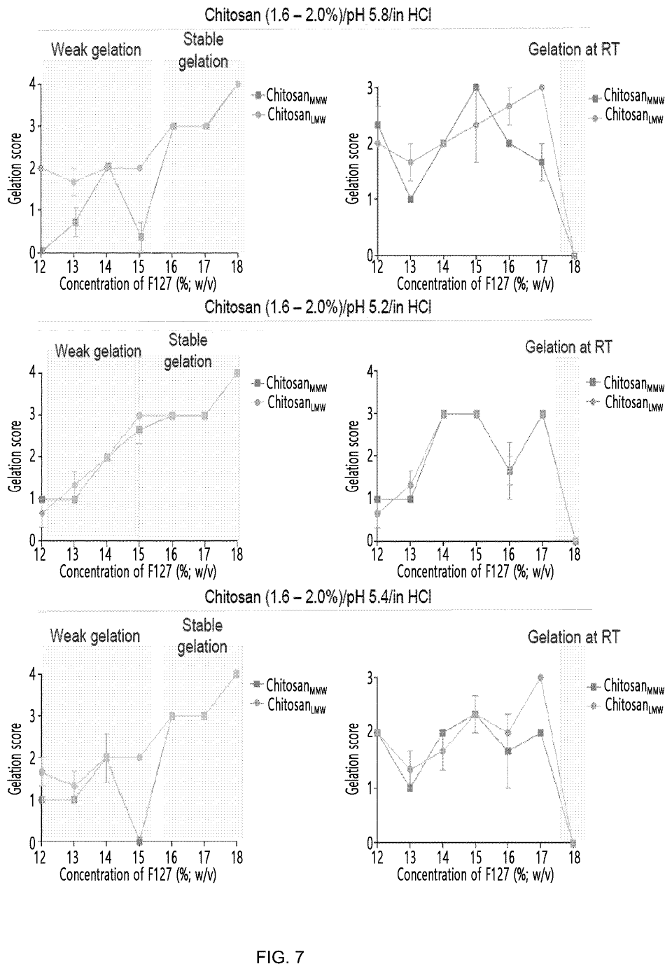

FIG. 7 is an evaluation of the physical properties of hydrogel according to the concentration of Pluronic F127, a temperature-sensitive polymer.

Pluronic F127 is very concentration-sensitive as it gels physically. As a result of an experiment in which the chitosan solution was sprayed on a hot plate with a surface of 37° C., when the concentration of F127 was lower than 16%, there was a tendency for weak gelation, such that it could not be fixed on the hot plate and flowed down. In contrast, when the concentration of F127 was 16% or more, excellent gelation performance was shown. However, when the concentration of F127 was 18%, it became a gel at room temperature, and spraying was impossible. Therefore, an appropriate concentration of F127 was found to be 16-17%.

Next, the temperature conditions for gel formation of the hydrogel were evaluated.

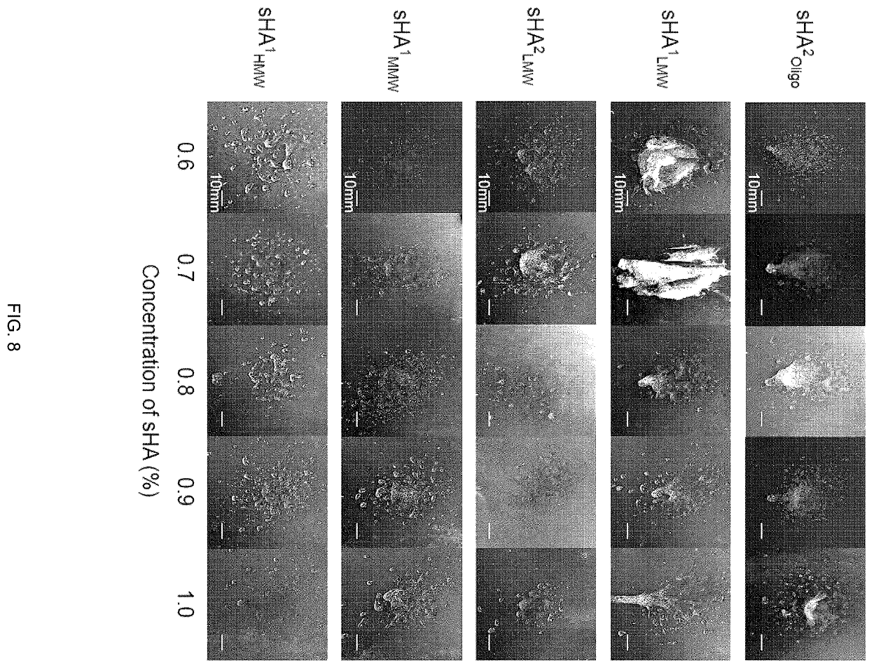

FIG. 8 is the result of spraying to form a hydrogel at conditions of 37° C.

After loading chitosan (2%)/F127 (16%) on one side of the dual spray device and sulfated hyaluronic acid (2%)/F127 (16%) on the other side, it was sprayed on a hot plate with a surface temperature of 37° C. The molecular weight of the sulfated hyaluronic acid used in the experiment was Oligo, LMW, MMW, and HMW, and the degree of sulfation was 1 or 2. As a result of the experiment, it was confirmed that sufficient complex formation with chitosan was possible even with a very small concentration of sulfated hyaluronic acid. Even under the condition of only 0.6% sulfated hyaluronic acid, it instantly gelled with chitosan, enabling stable attachment.

Since the advantage of the present invention is that the temperature-sensitive polymer is quickly gelled by electrostatic attraction before it is gelled by body temperature, thereby improving the attachment efficiency to the surgical site, the gelation reaction at room temperature is important. Accordingly, instead of performing the reaction of the two solutions under the condition of 37° C., the reaction was performed at room temperature (25° C.).

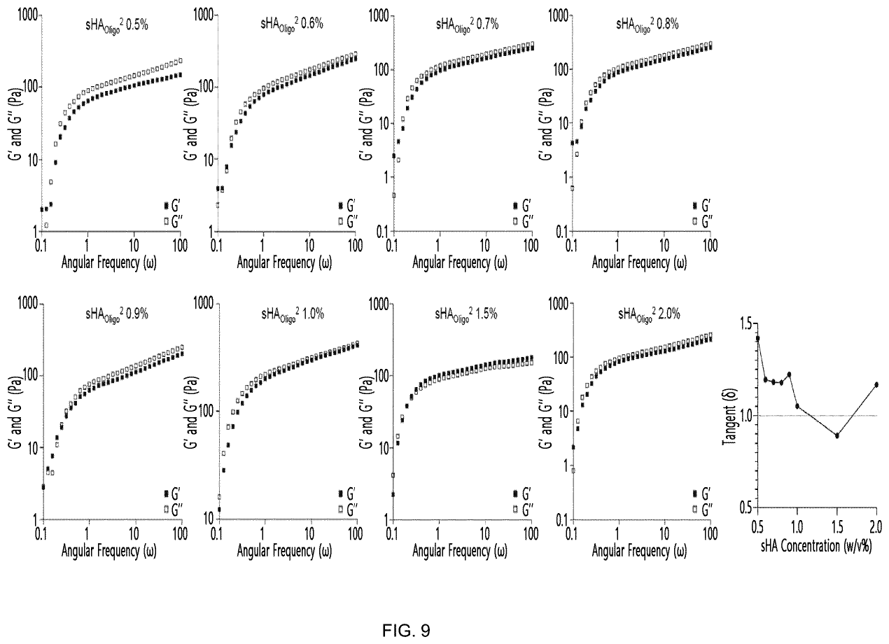

FIG. 9 is the result of spraying to form a hydrogel at 25° C.

Physical property analysis was carried out by way of a frequency sweep test. After loading the chitosan/F127 solution and the sulfated hyaluronic acid/F127 solution in halves into a rheometer and mixing the solutions, torsional stress was applied to the samples as the frequency sweep was performed. In particular, the torsion strength was the same (5% strain rate), and the frequency of torsion increased as time passed, such that the sample was twisted at a faster rate. In particular, the measured physical quantities were the storage modulus (G′) and the loss modulus (G″). If G′>G″, the elasticity is higher than the viscosity, showing more solid properties, and if G′<G″, the viscosity is higher than the elasticity, showing more liquid properties. The frequency sweep determines whether G′ or G″ is higher in the linear viscoelastic range, which usually appears in a section with a high frequency of torsion.

In FIG. 9, G′ and G″ showed almost the same values regardless of the concentration of sHA. Because of this, it does not exhibit better elasticity (G′>G″) as common hydrogels, but it is understood that the shape is easily deformed and exhibits a property of being easily attached to the surface, when stress is applied.

In addition, similar tendency was observed in sHA_{LMW}¹ and sHA_{LMW}².

While the present invention has been described with reference to the particular illustrative embodiments, it will be understood by those skilled in the art that various modifications and variation can be made in the present invention without departing from the spirit or scope of the invention as defined in the appended claims.

Accordingly, the scope of the present invention should be defined by the appended claims rather than the detailed description.

1. A method for preparing a hydrogel, comprising: bringing into contact with each other (i) a positively charged composition comprising a positively charged polymer or a derivative thereof, and a first temperature-sensitive polymer; and (ii) a negatively charged composition comprising a negatively charged polymer and a second temperature-sensitive polymer.

2. The method of claim 1, wherein the method further comprises spraying the positively charged composition and the negatively charged composition.

3. The method of claim 1 or 2, wherein the method further comprises gelling the positively charged composition, after bringing into contact with each other and spraying the positively charged composition and the negatively charged composition.

4. The method of claim 3, wherein the gelation is carried out in the body temperature range.

5. The method of any one of claims 1 to 4, the positively charged polymer comprises chitosan.

6. The method of claim 5, wherein the chitosan has a molecular weight of 50 kDa to 335 kDa.

7. The method of claim 5 or 6, wherein the chitosan has a molecular weight of 120 kDa to 250 kDa.

8. The method of any one of claims 5 to 7, wherein the positively charged composition comprises 1.2 wt. % to 2.0 wt. % chitosan.

9. The method of any one of claims 5 to 8, wherein the positively charged composition comprises 1.6 wt. % to 2.0 wt. % chitosan.

10. The method of any one or claims 1 to 9, wherein the positively charged composition further comprises hydrochloric acid (HCl).

11. The method of any one of claims 1 to 10, wherein the solvent provided to the positively charged composition is only hydrochloric acid (HCl).

12. The method of any one of claims 1 to 11, wherein the positively charged composition has an acidity of pH 4.7 to pH 6.0.

13. The method of any one of claims 1 to 12, wherein the positively charged composition has an acidity of pH 5.2 to pH 6.0.

14. The method of any one of claims 1 to 13, wherein the positively charged composition does not contain acetic acid.

15. The method of any one of claims 1 to 14, wherein the negatively charged polymer contains sulfated hyaluronate.

16. The method of any one of claims 1 to 15, wherein the negatively charged polymer comprises at least one sulfated hyaluronate selected from the following groups: 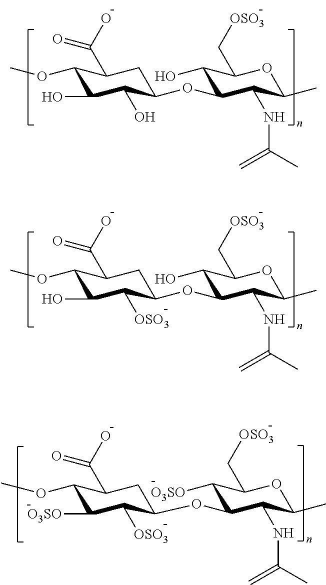
wherein n is a natural number from 20 to 5000.

17. The method of any one of claims 1 to 16, wherein the first temperature-sensitive polymer and/or the second temperature-sensitive polymer comprise a poloxamer.

18. The method of any one of claims 1 to 17, wherein the first temperature-sensitive polymer and/or the second temperature-sensitive polymer comprise a compound of the following structure: 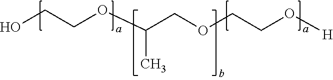
wherein a is a natural number from 2 to 130, and b is a natural number from 15 to 67.

19. The method of any one of claims 1 to 18, wherein the first temperature-sensitive polymer and/or the second temperature-sensitive polymer comprise a compound of the following structure:

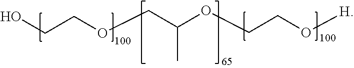

20. The method of any one of claims 1 to 19, wherein the first temperature-sensitive polymer and/or the second temperature-sensitive polymer have a molecular weight of 10 kDa to 15 kDa.

21. The method of any one of claims 1 to 20, wherein the positively charged composition comprises the first temperature-sensitive polymer in an amount of 16% to 17%, and/or the negatively charged composition comprises the second temperature-sensitive polymer in an amount of 16% to 17%.

22. The method of any one of claims 1 to 21, wherein the total amount of the first temperature-sensitive polymer and the second temperature-sensitive polymer contained in the hydrogel is less than 18%.

23. The method of any one of claims 1 to 22, wherein the contact is carried out at room temperature.

24. A method of treating a wound of a subject, comprising applying a hydrogel prepared by the method according to any one of claims 1 to 23 to the wound.

25. The method of claim 24, wherein the method further comprises spraying the hydrogel onto the wound.

26. The method of claim 24 or 25, wherein the wound is a surgical site.

27. The method of any one of claims 24 to 26, wherein the method treats inflammation of the wound.

28. The method of any one of claims 24 to 27, wherein the subject is a human.

29. A kit comprising: (i) a positively charged composition comprising a positively charged polymer or a derivative thereof, and a first temperature-sensitive polymer; and
(ii) a negatively charged composition comprising a negatively charged polymer and a second temperature-sensitive polymer.

30. The kit of claim 29, wherein the kit further comprises spraying a mixture of the positively charged composition and the negatively charged composition.

31. The kit of claim 29 or 30, wherein the positively charged composition undergoes gelation after contact and spraying.

32. The kit of claim 31, wherein the gelation is carried out in body temperature range.

33. The kit of any one of claims 29 to 32, wherein the positively charged polymer comprises chitosan.

34. The kit of claim 33, wherein the chitosan has a molecular weight of 50 kDa to 335 kDa.

35. The kit of claim 33 or 34, wherein the chitosan has a molecular weight of 120 kDa to 250 kDa.

36. The kit of any one of claims 33 to 35, wherein the positively charged composition comprises 1.2 wt. % to 2.0 wt. % chitosan.

37. The kit of any one of claims 33 to 36, wherein the positively charged composition comprises 1.6 wt. % to 2.0 wt. % chitosan.

38. The kit of any one of claims 29 to 37, wherein the positively charged composition further comprises hydrochloric acid (HCl).

39. The kit of any one of claims 29 to 38, wherein the solvent provided to the positively charged composition is only hydrochloric acid (HCl).

40. The kit of any one of claims 29 to 39, wherein the positively charged composition has an acidity of pH 4.7 to pH 6.0.

41. The kit of any one of claims 29 to 40, wherein the positively charged composition has an acidity of pH 5.2 to pH 6.0.

42. The kit of any one of claims 29 to 41, wherein the positively charged composition does not contain acetic acid.

43. The kit of any one of claims 29 to 42, wherein the negatively charged polymer contains sulfated hyaluronate.

44. The kit of any one of claims 29 to 43, wherein the negatively charged polymer comprises at least one sulfated hyaluronate selected from the following groups: 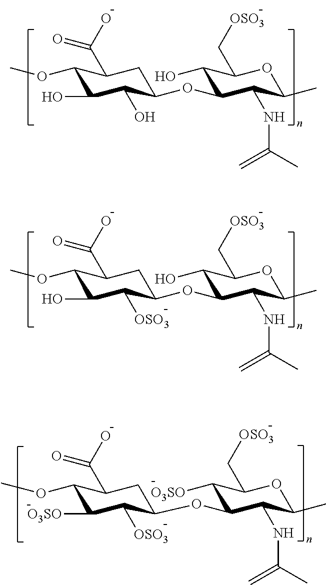
wherein n is a natural number from 20 to 5000.

45. The kit of any one of claims 29 to 44, wherein the first temperature-sensitive polymer and/or the second temperature-sensitive polymer comprise a poloxamer.

46. The kit of any one of claims 29 to 45, wherein the first temperature-sensitive polymer and/or the second temperature-sensitive polymer comprise a compound of the following structure: 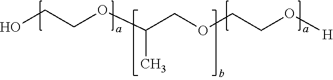
wherein a is a natural number from 2 to 130, and b is a natural number from 15 to 67.

47. The kit of any one of claims 29 to 46, wherein the first temperature-sensitive polymer and/or the second temperature-sensitive polymer comprise a compound of the following structure:

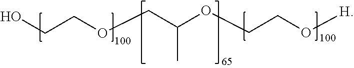

48. The kit of any one of claims 29 to 47, wherein the first temperature-sensitive polymer and/or the second temperature-sensitive polymer have a molecular weight of 10 kDa to 15 kDa.

49. The kit of any one of claims 29 to 48, wherein the total amount of the first temperature-sensitive polymer and the second temperature-sensitive polymer contained in the hydrogel is less than 18%.

50. The kit of any one of claims 29 to 49, wherein the positively charged composition comprises the first temperature-sensitive polymer in an amount of 16% to 17%, and/or the negatively charged composition comprises the second temperature-sensitive polymer in an amount of 16% to 17%.

51. The kit of any one of claims 29 to 50, wherein the hydrogel supports a physiologically active material.

52. Use of the kit according to any one of claims 29 to 51 in treatment of wounds in a subject.

53. Use of the kit according to any one of claims 29 to 51 in treatment of inflammation.
